(19) United States
(12) Patent Application Publication
(10) Pub. No.: US 2022/0234976 A1
(43) Pub. Date: Jul. 28, 2022

(54) MICROWAVE-ASSISTED DECARBOXYLATION OF ORGANIC ACIDS IN HEMP MATERIALS

(71) Applicant: S.S. Steiner, Inc., New York, NY (US)

(72) Inventors: John Paul Maye, Great Falls, VA (US); Jeremy Leker, Yakima, WA (US)

(21) Appl. No.: 17/611,643

(22) PCT Filed: May 14, 2020
(86) PCT No.: PCT/US20/32791

§ 371 Date: Nov. 16, 2021

Related U.S. Application Data
(60) Provisional application No. 62/848,982, filed on May 16, 2019.

(51) Int. Cl. C07C 37/56; C07D 311/80

(52) U.S. Cl. CPC C07C 37/56; C07D 311/80

(57) ABSTRACT

The present disclosure provides a method for decarboxylation of organic acids in hemp materials. The method includes placing hemp biomass in a container. The method further includes heating the hemp biomass using a microwave so that the hemp biomass receives between 100 J/g and 2,000 J/g, inclusive, total microwave energy. The method further includes decarboxylating the hemp biomass to form decarboxylated products.

### CROSS-REFERENCE TO RELATED APPLICATIONS

The present application claims the benefit of the following U.S. Provisional Application: U.S. Provisional Application Ser. No. 62/848,982, filed May 16, 2019, entitled MICROWAVE-ASSISTED DECARBOXYLATION OF ACIDS IN HEMP BIOMASS, which is incorporated herein by reference in the entirety.

### TECHNICAL FIELD

The present application generally relates to decarboxylation of organic acids, in particular, to decarboxylating organic acids in hemp materials, such as hemp biomass.

### BACKGROUND

Many hemp varieties are being bred for high concentrations of Cannabidiolic Acid (CBDA). Although CBDA is believed to have some medicinal value, its decarboxylated counterpart, Cannabidiol (CBD) is primarily the ingredient of interest. Current processes for CBDA decarboxylation in the hemp biomass to make CBD, however, require very high temperatures and long heating times. FIG. 1 shows the times and temperatures required to fully decarboxylate CBDA to CBD in the hemp biomass using a convection oven. At an industrial scale the hemp biomass can become flammable at temperatures over 140° C. making this a safety concern. In addition, full conversion of CBDA to CBD typically is not achieved unless temperatures of 135° C. are achieved and held for at least 60 minutes. Batch heating or continuous heating for 60 minutes uses a large amount of energy. There is a real need to decarboxylate the hemp biomass at a much faster and safer rate for large scale production.

### SUMMARY

A method for microwave-assisted decarboxylation organic acids in hemp materials is disclosed. In one or more embodiments, the disclosure provides a method of decarboxylation of an organic acid in hemp biomass includes heating hemp biomass using a microwave to a temperature for a set duration of time to decarboxylate at least between 80% and 99.9% of the organic acid.

The above method may be further characterized by one or more of the following additional features or steps, which may be combined with one another or any other portion of the description in this specification, including specific examples, unless clearly mutually exclusive:
(i) the organic acid is at least one of Cannabidiolic acid (CBDA), Tetrahydrocannabinolic acid (THCA), Cannabichromenic Acid (CBCA), and Cannabigerolic Acid (CBGA);
(ii) the method is a continuous process, wherein the microwave continuously heats the hemp biomass;
(iii) the method is an intermittent process, wherein the intermittent process is a process such that the microwave heats the hemp biomass at a first output energy for a first time duration and then at a different output energy for a second time duration;
(iv) the microwave is a belt driven continuous microwave oven;
(v) the first time duration is between 10 seconds and 60 seconds, inclusive;
(vi) the first time duration is between 10 second and 30 seconds, inclusive;
(vii) the method is a batch process, wherein a batch of the hemp biomass is placed in a container near the microwave, heated by the microwave, and then removed from the microwave;
(viii) the method is driven by a belt, wherein the belt moves the hemp biomass under the microwave;
(ix) the decarboxylation is conducted with mixing;
(x) the decarboxylation is conducted without mixing;
(xi) the temperature range to decarboxylate at least between 80% and 99.9% of the organic acid is between 120° C. and 160° C., inclusive; (xii) the temperature range to decarboxylate at least between 80% and 99.9% of the organic acid is between 135° C. and 140° C., inclusive;
(xiii) the set duration of time to decarboxylate at least between 80% and 99.9% of the organic acid is between 5 minutes and 60 minutes, inclusive;
(xiv) the microwave heats the hemp biomass with a heat density between 60 kW/m³ and 130 kW/m³, inclusive;
(xv) the microwave heats the hemp biomass with a heat density between 80 kW/m³ and 110 kW/m³, inclusive;
(xvi) the microwave heats the hemp biomass with a specific heat between 350 J/g and 1,750 J/g, inclusive;
(xvii) the microwave heats the hemp biomass with a specific heat between 600 J/g and 1,500 J/g, inclusive;
(xviii) cannabidiol (CBD) in the hemp biomass prior to the decarboxylation is between 1% and 40% by mass, inclusive;
(xix) the method further includes adding mineral additives to the hemp biomass prior to the heating; and
(xx) the mineral additives are at least one of salt form of sodium (Na), potassium (K), calcium (Ca), or magnesium (Mg).

The disclosure also provides a method for decarboxylation of an organic acid in hemp biomass includes placing the hemp biomass in a container. The method further includes heating the hemp biomass using a microwave so that the hemp biomass receives between 100 J/g and 2,000 J/g, inclusive, total microwave energy. The method also includes decarboxylating the hemp biomass to form decarboxylated products.

The above method may be further characterized by one or more of the following additional features or steps, which may be combined with one another or any other portion of the description in this specification, including specific examples, unless clearly mutually exclusive:
(i) wherein the organic acid is at least one of Cannabidiolic acid (CBDA), Tetrahydrocannabinolic acid (THCA), Cannabichromenic Acid (CBCA), and Cannabigerolic Acid (CBGA);
(ii) the decarboxylated products include at least one of Cannabidiol (CBD), Tetrahydrocannabinol (THC), Cannabichromane (CBC), and Cannabigerol (CBG);
(iii) the method is a continuous process, wherein the microwave continuously heats the hemp biomass;
(iv) wherein the method is an intermittent process, wherein the intermittent process is a process such that the microwave irradiates the hemp biomass at a first output energy for a first time duration and then at a different output energy for a second time duration;
(v) the first time duration is between 10 seconds and 60 seconds, inclusive;
(vi) the first time duration is between 10 second and 30 seconds, inclusive;
(vii) the method is a batch process, wherein a batch of the hemp biomass is placed in the container near the microwave, heated by the microwave, and then removed from the microwave;
(viii) the method is driven by a belt, wherein the belt moves the hemp biomass under the microwave;
(ix) the decarboxylation is conducted with mixing;
(x) the decarboxylation is conducted without mixing;
(xi) the microwave heats the hemp biomass with a heat density between 60 kW/m³ and 130 kW/m³, inclusive;
(xii) the microwave heats the hemp biomass with a heat density between 80 kW/m³ and 110 kW/m³, inclusive;
(xiii) the microwave heats the hemp biomass with a specific heat between 350 J/g and 1,750 J/g, inclusive;
(xiv) the microwave heats the hemp biomass with a specific heat between 600 J/g and 1,500 J/g, inclusive;
(xv) the CBD in the hemp biomass prior to the decarboxylation is between 1% and 40% by mass, inclusive;
(xvi) the method further includes adding mineral additives to the hemp biomass prior to the heating; and
(xvii) the mineral additives are at least one of salt form of sodium (Na), potassium (K), calcium (Ca), or magnesium (Mg).

### BRIEF DESCRIPTION OF THE DRAWINGS

Embodiments of the present disclosure are described by way of example in greater detail with reference to the attached figures, which are not necessarily to scale, and in which:

FIG. 1 is a table summarizing data for decarboxylation of cannabidiolic acid (CBDA) in hemp biomass in electric convection oven;

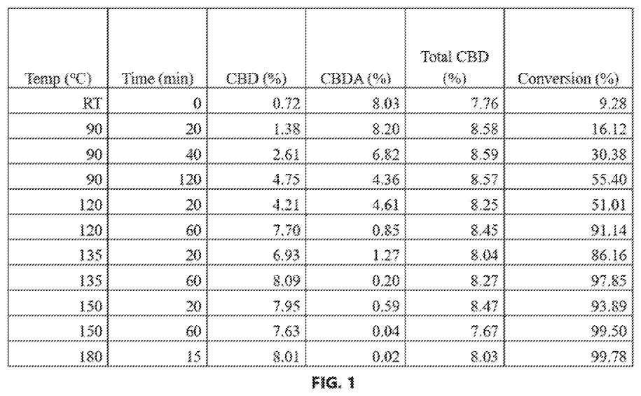

FIG. 2 is a flow chart illustrating a method of decarboxylation of organic acids in hemp biomass using a microwave, in accordance with one or more embodiments of this disclosure;

FIG. 3 is a schematic diagram of a setup for decarboxylation of organic acids in hemp biomass using a microwave, in accordance with one or more embodiments of this disclosure;

FIG. 4 is a table summarizing data for decarboxylation of organic acids in hemp biomass using a microwave, in accordance with one or more embodiments of this disclosure;

FIG. 5 is a table summarizing data for decarboxylation of organic acids in hemp biomass in a 1 ft³ reactor using a microwave, in accordance with one or more embodiments of this disclosure;

FIG. 6 is a table summarizing data for decarboxylation of organic acids in hemp biomass in a 10 ft³ reactor using a microwave, in accordance with one or more embodiments of this disclosure;

FIG. 7 is a table summarizing data for decarboxylation of organic acids in hemp biomass using a microwave, in accordance with one or more embodiments of this disclosure; and

FIG. 8 is a table summarizing data for decarboxylation of organic acids in hemp biomass using a microwave, in accordance with one or more embodiments of this disclosure.

### DETAILED DESCRIPTION OF THE INVENTION

As used herein, and unless noted the contrary, the following terms and phrases have the meaning noted below:

“Hemp” refers to a strain of the Cannabis sativa plant species.

“Hemp biomass” refers to the whole hemp plant or specific parts of the hemp plant, particularly the leaves, buds, stalk, or combinations thereof.

“Hemp fiber biomass” refers to organic materials of the hemp plant that are left over.

“Hemp powder” refers to powder produced by grinding hemp biomass.

“Hemp pellet” refers to a pressed form produced by pressing the hemp powder.

“Cannabinoids” refer to chemicals found in Cannabis including cannabigerol-, cannabichromene-, cannabidiol-, tetrahydrocannabinol-, cannabinol-, cannabielsoin-, iso-tetrahydrocannabinol-, cannabicyclol-, and cannabicitran-types.

“Cannabinoid acids” refer to chemicals found in Cannabis including Cannabidiolic acid, Cannabichromanic acid, Tetrahydrocannabinolic acid, and Cannabigerolic acid.

“Carboxylic acid” refers to an organic compound that contains at least one carboxyl group [(—C(═O)OH) or (—COOH)].

“Alcohol” refers to an organic compound that contains at least one hydroxyl group (—OH).

“Decarboxylation” refers to a chemical reaction that removes a carboxyl group and releases carbon dioxide, thereby, for example, converting an organic acid having a hydroxyl group to a corresponding alcohol.

“Conversion” refers to a percentage of reactants converted to products in a chemical reaction.

“Yield percent” refers to a percentage of pure product yielded divided by pure product expected in a chemical reaction.

“RT” refers to room temperature.

“N/A” refers to not applicable.

“Dry basis” refers to an expression that neglects presence of water in calculations. For example, when 10% organic compound and 10% moisture are present, a concentration of the organic compound on a dry basis would be 10/(1-0.1)=11.1%.

“Energy density” refers to amount of energy stored in a given system per unit volume.

“Specific energy” refers to amount of energy stored in a given system per unit mass.

“Cannabidiolic acid” is also known as 2,4-dihydroxy-3-[(1R,6R)-3-methyl-6-prop-1-en-2-ylcyclohex-2-en-1-yl]-6-pentylbenzoic acid, cannabidiol acid or CBDA. Its chemical formula is C₂₂H₃₀O₄.

“Cannabidiol” is also known as 2-[(1R,6R)-6-isopropenyl-3-methylcyclohex-2-en-1-yl]-5-pentylbenzene-1,3-diol, cannabidiolum or CBD. Its chemical formula is C₂₁H₃₀O₂.

“Cannabichromanic acid” is also known as CBCA, 2-COOH—CBC, or CBC—COOH. Its chemical formula is C₂₂H₃₀O₄.

“Cannabichromene” is also known as 2-methyl-2-(4-methylpent-3-enyl)-7-pentyl-5-chromenol, cannabichrome, or CBC. Its chemical formula is C₂₁H₃₀O₂.

“Tetrahydrocannabinolic acid” is also known as (6aR,10aR)-1-hydroxy-6,6,9-trimethyl-3-pentyl-6a,7,8,10a-tetrahydro-6H-benzo[c]chromene-2-carboxylic acid, 2-carboxy-THC, THCA, or 2-COOH-THC. Its chemical formula is C₂₂H₃₀O₄.

“Tetrahydrocannabinol” is also known as (6aR, 10aR)-6,6,9-trimethyl-3-pentyl-6a,7,8,10a-tetrahydro-6H-benzo[c]chromen-1-ol, (6aR,10aR)-delta-9-tetrahydrocannabinol or THC. Its chemical formula is C₂₁H₃₀O₂.

“Cannabigerolic acid” is also known as 3-[(2E)-3,7-dimethylocta-2,6-dienyl]-2,4-dihydroxy-6-pentylbenzoic acid or CBGA. Its chemical formula is C₂₂H₃₀O₄.

“Cannabigerol” is also known as 2-[(2E)-3,7-dimethyocta-2,6-dienyl]-5-pentyl-benzene-1,3-diol or CBG. Its chemical formula is C₂₁H₃₂O₂.

Referring now to the description and drawings, example embodiments of the disclosed apparatuses, systems, and methods are shown in detail.

The present disclosure is directed to a decarboxylation method using a microwave. More particularly, embodiments of the present disclosure are directed to a decarboxylation of hemp materials such as hemp biomass using a microwave to convert organic acids into corresponding alcohols.

The hemp biomass includes stalks, leaves, and flowers. The hemp biomass contains a variety of chemical compounds. For example, the hemp biomass may include organic acids such as Cannabidiolic acid (CBDA), Tetrahydrocannabinolic acid (THCA), Cannabichromanic acid (CBCA), Cannabigerolic acid (CBGA), and the like. The average organic acid content in the hemp biomass ranges from 1% to 20% by weight on a dry basis. The organic acids in the hemp biomass can be further processed to form chemicals for medicinal purposes, dietary supplement, cosmetics, and any combinations thereof.

The organic acids mentioned above in the hemp biomass usually are decarboxylated into the corresponding alcohols. For example, decarboxylating Cannabidiolic Acid (CBDA) yields Cannabidiol (CDB), decarboxylating Tetrahydrocannabinolic acid (THCA) yields Tetrahydrocannabinol (THC), decarboxylating Cannabichromanic acid (CBCA) yields Cannabichromane (CBC), and Cannabigerolic acid (CBGA) yields Cannabigerol (CBG).

Decarboxylation is a chemical reaction that removes a carboxyl group [(C(═O)OH) or (—COOH)] and releases carbon dioxide (CO₂), which is a replacement of a carboxyl group (—COOH) with a hydrogen atom. The reaction starts when a chemical with a carboxyl group (—COOH) is placed in decarboxylation conditions, which yields 1 equivalent of carbon dioxide and 1 equivalent of the corresponding chemical with a hydrogen. Most decarboxylation reactions involve a radical reaction and such a reaction may include a Barton decarboxylation, a Kolbe reaction, a Kochi reaction, a Hunsdiecker reaction, or any combinations thereof. Some decarboxylation reactions do not involve a radical reaction such as Tsuji-Trost reaction, a Krapcho decarboxylation, or a combination of these reactions. Both types of decarboxylation, those involving a radical reaction and those not involving a radical reaction, may be used in the present disclosure, and may be combine with one another in any combinations.

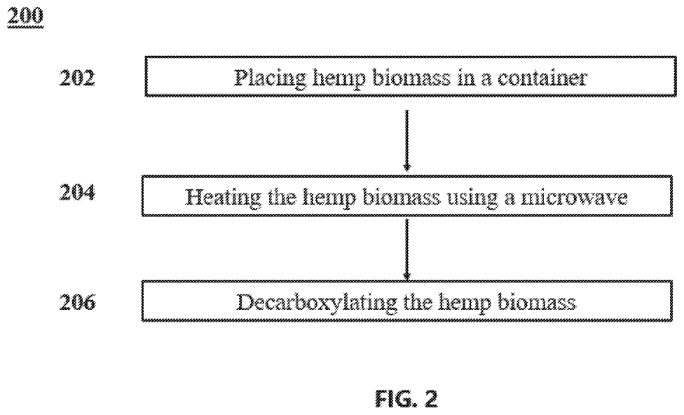

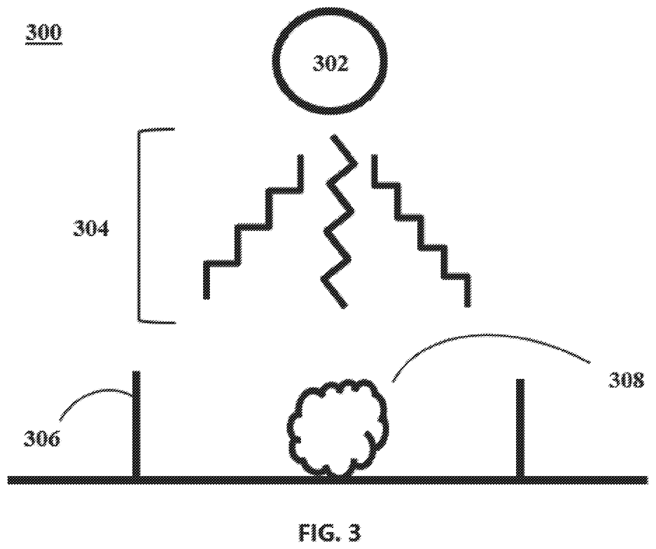

Now referring to FIGS. 2-3, FIG. 2 is a flow chart illustrating a method of decarboxylation of organic acids in hemp biomass using a microwave. FIG. 3 is a schematic diagram of a setup 300 for decarboxylation of organic acids in hemp biomass using a microwave. The hemp biomass 308 is placed in a container 306 in step 202. The container 306 for the hemp biomass may come in various forms. For example, the container 306 may include a dish, a bowl, a flask, a beaker, a test tube, a reactor, a reaction vessel, a vial, or a flat surface, such as a conveyer belt, or any vessel conventionally used to hold materials being exposed to microwave irradiation 304 from an industrial microwave, or any combinations thereof. It is noted that any vessel that can hold solids may be appropriate because the hemp biomass is solid form.

It is noted that, while embodiments of the present disclosure may be described using only one container, such a configuration is merely discussed for illustrative purposes. Embodiments of the present disclosure may use on or more additional containers, for example to hold excess hemp biomass which can be periodically fed to the main container that is microwave irradiated by the microwave. For example, the additional container may hold and feed the hemp biomass onto a conveyer belt which moves under the microwave. In this regard, the conveyer belt is the primary container and the additional container acts as a secondary container. By way of another example, multiple containers holding the hemp biomass may be used and each container may rotate position after microwave irradiation by the microwave is complete

The hemp biomass 308 may contain organic acids such as Cannabidiolic acid (CBDA), Tetrahydrocannabinolic acid (THCA), Cannabichromanic acid (CBCA), Cannabigerolic acid (CBGA), and the like. The average organic acid content in the hemp biomass ranges from 1% to 20% by weight on a dry basis.

The hemp biomass 308 may come in various forms. For example, the hemp biomass 308 used in the present disclosure may be a hemp powder. Hemp powder is fine powder produced by grinding the hemps including hemp, hemp biomass, hemp fiber biomass. By way of another example, the hemp biomass used in the present disclosure may be a hemp pellet.

The hemp biomass 308 may contain decarboxylated products, for example CBD, THC, and CBC, prior to decarboxylation formed when drying the plant after harvest. The percentage of the decarboxylated products in the hemp biomass 308 depends farming process conditions and drying conditions. For example, the percentage of the decarboxylated products in the hemp biomass 308 prior to decarboxylation may be between 0.01% and 50% by mass, between 0.1% and 45% by mass, or between 1% and 40% by mass, inclusive. When the hemp biomass 308 undergoes decarboxylation, the percentage of the decarboxylated products may be increased.

The hemp biomass 308 used in the present disclosure is dried prior to decarboxylation, for example, by a Kiln drying, and may contain moisture. The moisture is the presence of liquid such as water. The content of moisture depends on conditions of farming processes, climate, and drying conditions. For example, the content of hemp biomass moisture may be between 0.5% and 50% by mass, between 1% and 25% by mass, or between 5% and 10% by mass, inclusive. When the hemp biomass 308 undergoes decarboxylation, some of the moisture content may be decreased.

In one embodiment, a microwave 302 irradiates the hemp biomass 308 placed in the container 306 in step 204. The hemp biomass 308 is exposed to a microwave irradiation 304 of the microwave 302 during decarboxylation. The container 306 may be placed below the microwave 302 such that the hemp biomass 308 receives the microwave irradiation 304 evenly. The container 306 and the microwave 302 may be integrated together to microwave irradiate the hemp biomass more efficiently.

The methods of the present disclosure may allow the hemp biomass 308 to receive microwave irradiation 304 evenly. In general, chemical reactions may be performed while mixing, for example by stirring to maintain homogeneity of a reaction mixture. The mixing increases reaction rate of chemical reactions because more surface area of the hemp biomass is exposed to the microwave irradiation by mixing and mixing breaks down the hemp biomass into smaller pieces which necessarily have a higher surface area to volume ratio. In order to increase decarboxylation conversion, mixing may be used within the container to expose the hemp biomass evenly to microwave irradiation because the microwave may not provide the microwave irradiation evenly throughout the container. For example, mixing may include mechanical stirring such as a stirring paddle, use of a stirring magnet, or the combination thereof. The mixing may be via a continuous movement or via intermittent movements. The mixing speed may vary and may be adjustable.

It is noted that mixing is not always necessary in methods of the present disclosure. The same result or satisfactory results can be obtained with different methods without mixing. A conveyor belt may move the hemp biomass through a microwave tunnel in a continuous process without mixing. Alternatively, a conveyer belt may move and stop at a periodic interval in an intermittent process. Compared to the continuous process, the intermittent process may better control the amount of time that the hemp biomass is microwave irradiated. The time spent under the microwave is adjustable based on the intervals and the speed of the conveyer movement. In another alternative, a batch of hemp biomass may be placed in a container near the microwave, then subjected to microwave irradiation, and then removed from the microwave in a batch process. The entire container, if mobile may be removed, or the hemp biomass may be removed from a fixed container near the microwave.

In some examples, additives may be added to the hemp biomass to increase the rate of decarboxylation. For example, the additives may include mineral additives such as salt forms of sodium (Na), potassium (K), calcium (Ca), magnesium (Mg), or any combination thereof. The additives may be in an aqueous liquid form which can be sprayed over the hemp biomass prior to decarboxylation. Further, the additives may be in a solid form which can be sprinkled over, placed under, or mixed into, or any combinations thereof with respect to the hemp biomass prior to decarboxylation.

Microwave irradiation is a form of electromagnetic radiation with a frequency above that of radio waves and below that of infrared light, between 300 GhZ and 300 mHz. The wavelength of microwaves is between 1 mm and 1 m. The microwave used to decarboxylate cannabinoids acids may be an industrial microwave. The industrial microwaves have more capabilities than microwaves used for cooking purposes. Output energy of most kitchen microwaves is between 1,000-1,200 W. An industrial microwave may output higher energy, such as up to 300 kW, so that it can provide high energy densities at a core location. The amount of microwave irradiation applied to the hemp biomass in methods of the present disclosure is a function of the size, number, and an output energy of microwaves. Larger microwave provides more microwave irradiation. Multiple microwaves provide more microwave irradiation. A higher output energy from the microwave provides more microwave irradiation.

The hemp biomass may be microwave irradiated with a specified energy density. For example, the output energy from the microwave in the example 2 is set at 3 kW for a 1 ft³ reactor and the energy density in this case is 105.9 kW/m³. By way of another example, the output energy from the microwave in the example 3 is set at 25 kW for a 10 ft³ reactor and the energy density in this case is 88.28 kW/m³. The hemp biomass may be microwave irradiated with an energy density between 40 kW/m³ and 150 kW/m³, between 60 kW/m³ and 130 kW/m³, or between 80 kW/m³ and 110 kW/m³, inclusive.

The hemp biomass may be microwave irradiated with a specific energy. For example, the hemp biomass may be microwave irradiated with the specific energy between 100 J/g and 2,000 J/g, between 350 J/g and 1,750 J/g, or between 600 J/g and 1,500 J/g, inclusive.

In some methods of the present disclosure, the output energy of the microwave is fixed. For example, the output energy may be determined prior to the decarboxylation and the hemp biomass may be exposed to continuous output energy from the microwave. In this sense, the hemp biomass is exposed to the same microwave irradiation throughout decarboxylation.

In some embodiments, the output energy of the microwave is intermittent. For example, the output energy of the microwave may vary in such a way that the microwave irradiates the hemp biomass at a first output energy for a first time duration and then at a different output energy for a second time duration, such that the hemp biomass is not exposed to the same output energy throughout decarboxylation. Further, embodiments of the present disclosure allow the gradual output energy increase and/or decrease of the microwave to achieve better yields and conversions.

The microwave may microwave irradiate the hemp biomass in such a way that decarboxylation of at least one organic acid in the hemp biomass proceeds efficiently. For instance, the hemp biomass may be microwave irradiated such that it is heated to a temperature range between 80° C. and 200° C., between 100° C. and 180° C., or between 120° C. and 160° C., inclusive. Decarboxylation of organic acids in hemp biomass is typically efficient at a temperature range between 135° C. and 140° C. This temperature range may be maintained for a set duration of time, such as between 1 minute and 25 minutes, between 3 minutes and 20 minutes, or between 5 minutes and 15 minutes, inclusive, for example by further microwave irradiation. High temperatures, such as above 220° C., may result in the hemp biomass burning, and may, therefore, be avoided.

The microwave may operate continuously or in a pulsed fashion. For example, the microwave may provide microwave irradiation for a set duration of time, such as between 10 seconds and 60 seconds, inclusive, or between 10 second and 30 seconds, inclusive, then cease providing microwave irradiation for a set duration of time, such as between 1 second and 60 seconds, inclusive, or between 1 second and 30 seconds, inclusive, in an alternating fashion. It is noted that, while certain time durations are mentioned, these are merely provided for illustrative purposes. Any duration may be suitable depending on the method.

In some examples, the time that the hemp biomass is microwave irradiated may be between 1 minute and 100 minutes, between 3 minutes and 80 minutes, or between 5 minutes and 60 minutes, inclusive.

Although methods using only one microwave are described in the description, such a configuration is merely provided for illustrative purposes. Multiple microwaves may be used for one decarboxylation process. For example, two or more microwaves may be used at a certain distance apart in a sequential decarboxylation. Further, multiple microwaves may provide different powers so that the decarboxylation of the hemp biomass may reach higher conversion. For instance, a first microwave operating at one power, such as a high power, may be used to quickly heat up the hemp biomass to between 135° C. and 140° C., while a second microwave operating at a second power, such as a lower power, may maintain the temperature of the hemp biomass. Additionally, the hemp biomass may be sandwiched between two microwaves receiving microwave irradiation both from top and bottom.

The decarboxylation forms at least one decarboxylated product. For example, decarboxylation of organic acids in the hemp biomass may forms alcohols. More specifically, decarboxylating Cannabidiolic acid (CBDA) forms Cannabidiol (CDB), decarboxylating Tetrahydrocannabinolic acid (THCA) forms Tetrahydrocannabinol (THC), decarboxylating Cannabichromanic acid (CBCA) forms Cannabichromane (CBC), and Cannabigerolic acid (CBGA) forms Cannabigerol (CBG). When CBDA, THCA, CBCA, and CBGA are decarboxylated to form their corresponding alcohols, a carboxyl group (—COOH) is replaced with a hydrogen atom (—H) in addition to existing hydroxyl groups (—OH) so decarboxylating these organic acids leads to the corresponding alcohols (chemical structure with a hydroxyl group). Although the present disclosure focuses on CBDA, THCA, CBCA, and CBGA, such organic acids are merely presented for illustrative purposes. Other organic acids present in the hemp biomass may undergo decarboxylation to their corresponding alcohols. Thus, methods of the present disclosure may be used to decarboxylate any organic acid containing a with carboxyl group in the hemp biomass.

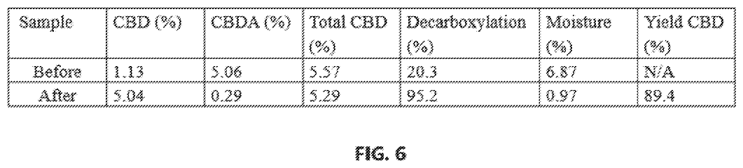

Method of the present disclosure may convert at least one organic acid in the hemp biomass into its corresponding alcohol with high conversion. For example, the conversion of at least one organic acid in the hemp biomass to its corresponding alcohol may be at least 80% and 99.9%, 90% and 99.9%, or 95% and 99.9%, inclusive. If more than one organic acid in the hemp biomass is converted to its corresponding alcohol by a method of the present disclosure total conversion of all such organic acids, or a particular set of such organic acids may be between 80% and 99.9%, 90% and 99.9%, or 95% and 99.9%, inclusive. Conversion efficiency of the decarboxylation may differ between different organic acids due to differences in reactivity of different organic acids in the decarboxylation. In general, the conversion efficiency of CBDA into CBD may be higher than the total conversion efficiency of other cannabinoid acids. For example, if the conversion efficiency of CBDA into CBD is 93.98%, the conversion efficiency of all other cannabinoid acids may be only 91.94%, as shown in FIG. 6. The concentration of cannabinoid acids other than CBDA in hemp biomass is much lower than the concentration of CBD or CBDA, as shown in FIG. 6 where CBDA is 6.22%, THCA is 0.23%, and CBCA is 0.31% prior to decarboxylation.

The decarboxylation step in methods of the present disclosure may also reduce moisture content in the hemp biomass. For example, the moisture content in the hemp biomass prior to the decarboxylation step may be between 0.5% and 50%, between 1% and 25%, or between 1% and 10%, inclusive. The moisture content in the hemp biomass after the decarboxylation step may be between 0.1% and 6.0%, between 0.2% and 3.0%, or between 0.25% and 1.5%, inclusive.

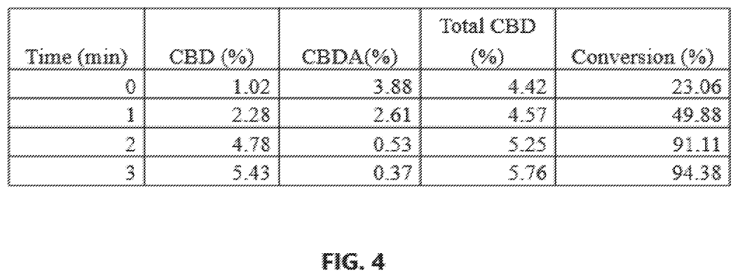

FIGS. 4-8 are tables summarizing data for decarboxylation of organic acids in hemp biomass using a microwave, in accordance with one or more embodiments of this disclosure. The following examples are provided to further illustrate the principles and specific aspects of the disclosure. They are not intended to and should not be interpreted to encompass the entire breath of all aspects of the disclosure.

### Example 1: Conversion Efficiency of CBDA to CBD

5 grams of hemp powder is placed into a kitchen microwave and microwave irradiated with 30 second pulses with mixing between the pulses. Samples are taken every minute and analyzed to measure the conversion efficiency of decarboxylation. Results are presented in FIG. 4. Data shown in FIGS. 4-8 for CBD and CBDA are indicated by mass percent (%). The total CBD (%) in FIGS. 4-8 is a total CBD taking into account of the carbon dioxide loss in mass upon decarboxylation. Molecular weight of CBD and CBDA are 314.46 g/mol and 358.47 g/mol, respectively. Decarboxylation removes a carboxyl group, adds a hydrogen atom in place of the carboxyl group, and releases a carbon dioxide. Thus, CBD total % is equal to a sum of CBD and 0.877*CBDA. The inventor observed that high conversions of decarboxylation are achieved after only 2 minutes, which is very unexpected as the hemp biomass is a mixture of various compounds. In general, chemical reactions of mixed components like the hemp biomass are expected to proceed slowly and leads to lower yields because of side reactions that may take place among all the components.

### Example 2: Decarboxylation of Organic Acids in Hemp Biomass in a 1 ft³ Reactor Using a Microwave

6.325 kg of hemp biomass is loaded into a 1 ft³ reactor with a microwave. The hemp biomass is mixed and exposed to 3 kW of the microwave irradiation from the microwave for 26 minutes after which the microwave irradiation was maintained a temperature of 135° C. for an additional 10 minutes. 5.91 kg of decarboxylated product is collected and cooled to ambient temperature.

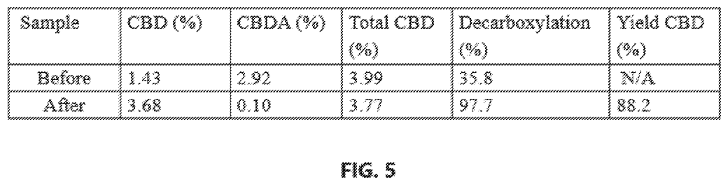

The decarboxylated product is analyzed via high performance liquid chromatography (HPLC) and the results are presented in FIG. 5. Samples of the hemp biomass are collected and then grinded into a powder with a blender. 2.500+−0.02 g of the samples are placed into a boston round bottle and 50 mL of 9:1 Methanol:Chloroform (volume:volume) is added. The round bottle is placed on an orbital shaker for 30 minutes. The round bottle is centrifuged at 4000 rpm for 4 minutes. 5 mL of supernatant is transferred to a 50 mL volumetric flask and made up to the volume in methanol. The sample solution is then filtered before injection into HPLC. HPLC Method: Mobile Phase A: 0.1% formic acid, 5 mM ammonium formate in Water Mobile. Phase B: 0.1% formic acid in Methanol 0.6 mL/min 57B:33A isocratic. Column: Restec ARC-18 1.8 um 50×2.1 mm. Column Oven 40° C. Injection 3 uL.

The reason why the total CBD % before is higher than the total CBD % after decarboxylation is that the conversion of decarboxylation is not 100%. The total CBD % in FIG. 5 factors in both the conversion of the decarboxylation and CBD content. It's noted that the yield of CBD is based on CBD out divided by total CBD in (3.68/3.99=0.882).

### Example 3: Decarboxylation of Organic Acids in Hemp Biomass in a 10 ft³ Reactor Using a Microwave

127 kg of hemp biomass is loaded into a 10 ft³ reactor with a microwave. The hemp biomass is mixed and microwave irradiated with 25 kW of output energy from the microwave until a temperature of 135° C. is achieved, for example, 45 minutes. The hemp biomass is maintained a temperature of 135° C. for 15 minutes before the hemp biomass is unloaded for a cooling. The hemp biomass is analyzed by HPLC and the results are presented in FIG. 6.

### Example 4: Decarboxylation of Organic Acids in Hemp Biomass Using a Microwave

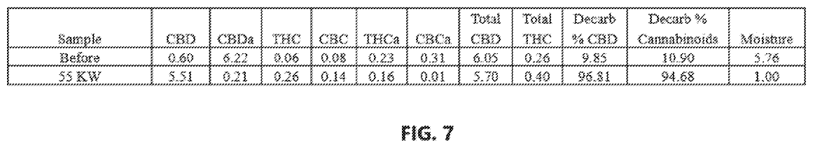

907 kg of hemp pellets is loaded into a container placed on top of a 4-foot-wide belt conveyor moving at a speed of 20 inches per minute. The hemp pellets are layered onto the conveyor belt ½ inch high. The conveyor belt passes through a 15-foot-long, 4-feet-wide microwave with output energy set at 30 kW and slowly increased to 60 kW until the temperature reaches 130° C. Samples are collected. Then, the output energy is lowered to 55 kW to maintain temperature until the end of the experiment. The results are presented in FIG. 7. The THC total % takes into account of carbon dioxide loss as explained above with CBD total % in FIG. 4. Thus, THC total % is equal to a sum of THC and 0.877*THCA. Decarb % CBD indicates the progress of the decarboxylation and is calculated by the formula (CBD/total CBD)×100.

### Example 5: Decarboxylation of Organic Acids in Hemp Biomass in a 1 ft³ Reactor Using a Microwave

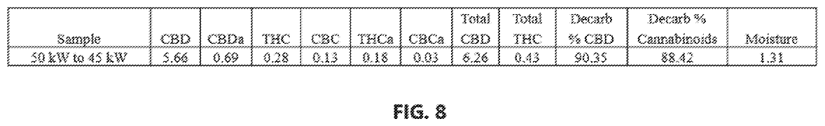

907 kg of hemp pellets is loaded into a container placed on top of a 4-foot-wide conveyor belt moving at a speed of 20 inches per minute. The hemp pellets are layered onto the conveyor belt ½ inch high. The conveyor belt passes through a 15-foot-long, 4-feet-wide microwave with output energy set at 50 kW. As the hemp pellets just starts to leave the microwave some minor smoking is observed so the output energy is lowered to 45 kW and kept the same output energy until the end of the experiment. The results are presented in FIG. 8.

Although this disclosure has been described in terms of certain embodiments, modifications (such as substitutions, additions, alterations, or omissions) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, and the operations of the systems and apparatuses may be performed by more, fewer, or other components. As another example, modifications may be made to the methods disclosed herein. The methods may include more, fewer, or other steps, and the steps may be performed in any suitable order.

What is claimed is:

1. A method for decarboxylation of an organic acid in hemp biomass, the method comprising:
heating hemp biomass using a microwave to a temperature range for a set duration of time to decarboxylate at least between 80% and 99.9% of the organic acid;
wherein the microwave heats the hemp biomass with a heat density between 60 kW/m³ and 130 kW/m³, inclusive.

2. The method of claim 1, wherein the organic acid is at least one of Cannabidiolic acid (CBDA), Tetrahydrocannabinolic acid (THCA), Cannabichromenic Acid (CBCA), and Cannabigerolic Acid (CBGA).

3-20. (canceled)

21. A method for decarboxylation of an organic acid in hemp biomass, comprises:
placing the hemp biomass in a container;
heating the hemp biomass using a microwave so that the hemp biomass receives between 100 J/g and 2,000 J/g, inclusive, total microwave energy; and
decarboxylating the hemp biomass to form decarboxylated products.

22. The method of claim 21, wherein the organic acid is at least one of Cannabidiolic acid (CBDA), Tetrahydrocannabinolic acid (THCA), Cannabichromenic Acid (CBCA), and Cannabigerolic Acid (CBGA).

23. The method of claim 21, wherein the decarboxylated products include at least one of Cannabidiol (CBD), Tetrahydrocannabinol (THC), Cannabichromane (CBC), and Cannabigerol (CBG).

24. The method of claim 21, wherein the method is a continuous process, wherein the microwave continuously heats the hemp biomass.

25. The method of claim 21, wherein the method is an intermittent process, wherein the intermittent process is a process such that the microwave irradiates the hemp biomass at a first output energy for a first time duration and then at a different output energy for a second time duration.

26. The method of claim 25, wherein the first time duration is between 10 seconds and 60 seconds, inclusive.

27. The method of claim 26, wherein the first time duration is between 10 second and 30 seconds, inclusive.

28. The method of claim 21, wherein the method is a batch process, wherein a batch of the hemp biomass is placed in the container near the microwave, heated by the microwave, and then removed from the microwave.

29. The method of claim 21, wherein the method is driven by a belt, wherein the belt moves the hemp biomass under the microwave.

30. The method of claim 21, wherein the decarboxylation is conducted with mixing.

31. The method of claim 21, wherein the decarboxylation is conducted without mixing.

32. The method of claim 21, wherein the microwave heats the hemp biomass with a heat density between 60 kW/m³ and 130 kW/m³, inclusive.

33. The method of claim 32, wherein the microwave heats the hemp biomass with a heat density between 80 kW/m³ and 110 kW/m³, inclusive.

34. The method of claim 21, wherein the microwave heats the hemp biomass with a specific heat between 350 J/g and 1,750 J/g, inclusive.

35. The method of claim 34, wherein the microwave heats the hemp biomass with a specific heat between 600 J/g and 1,500 J/g, inclusive.

36. The method of claim 23, wherein the CBD in the hemp biomass prior to the decarboxylation is between 1% and 40% by mass, inclusive.

37. The method of claim 21, further comprising:
adding mineral additives to the hemp biomass prior to the heating.

38. The method of claim 37, wherein the mineral additives are at least one of salt form of sodium (Na), potassium (K), calcium (Ca), or magnesium (Mg).
